(19) United States
(12) Patent Application Publication
(10) Pub. No.: US 2011/0044996 A1
(43) Pub. Date: Feb. 24, 2011

(54) NEUTRALIZING ANTIBODIES

(76) Inventors: Charles Mackay, New South Wales (AU); David Zahra, New South Wales (AU)

Correspondence Address: Pearl Cohen Zedek Latzer, LLP, 1500 Broadway, 12th Floor, New York, NY, 10036, US

(21) Appl. No.: 12/601,514

(22) PCT Filed: May 23, 2008
(86) PCT No.: PCT/AU08/00728

§ 371 Date: Jul. 30, 2010

Related U.S. Application Data
(60) Provisional application No. 60/931,402, filed on May 23, 2007.

(30) Foreign Application Priority Data
May 23, 2007 (AU) 2007902751

(51) Int. Cl. A61K 39/395; C07K 16/24; A61P 29/00; A61P 37/02; A61P 11/06; A61P 25/00

(52) U.S. Cl. 424/158.1; 530/389.2; 530/387.3; 530/388.23

(57) ABSTRACT

The present invention provides antibodies that bind, interact or otherwise associate with GM-CSF or a fragment, portion or part thereof and antagonize or neutralize GM-CSF activity. In accordance with embodiments of the present invention humanized monoclonal antibodies are generated which bind to human GM-CSF with high affinity and which inhibit the activity of GM-CSF.

### FIELD OF THE INVENTION

The present invention relates generally to antibodies that bind to granulocyte-macrophage colony stimulating factor (GM-CSF). More particularly the invention relates to high affinity, neutralizing humanized monoclonal antibodies specific for GM-CSF. The invention also relates to uses of such antibodies in the treatment or prevention of GM-CSF-mediated and GM-CSF-associated diseases or conditions. The invention further relates to methods for modulating GM-CSF-mediated and GM-CSF-associated diseases or conditions by the administration of antibodies of the invention.

### BACKGROUND OF THE INVENTION

Granulocyte-macrophage colony stimulating factor (GM-CSF) is a hematopoietic growth factor which regulates the differentiation, proliferation and function of granulocytes and monocytic cells such as macrophages.

GM-CSF is also a potent inflammatory cytokine, the activity or overexpression of which can have significant detrimental effects. GM-CSF is implicated in a variety of autoimmune and inflammatory diseases including rheumatoid arthritis, asthma, multiple sclerosis and idiopathic thrombocytopenic purpura. In asthma and rheumatoid arthritis, elevated levels of GM-CSF have been detected and correlated with the inflammatory process, whilst in experimental autoimmune encephalomyelitis, an animal model of multiple sclerosis, GM-CSF knockout mice are protected against the onset of the disease.

Accordingly, there is a clear need for the development of effective approaches to target GM-CSF and block or neutralize GM-CSF activity. Antibodies against GM-CSF offer one particularly suitable alternative as antagonists of GM-CSF with clear therapeutic applications, such as in the treatment or prevention of autoimmune or inflammatory conditions.

Whilst antibodies against GM-CSF are known in the art, there remains a need for the development of improved antibodies, for example with enhanced binding affinity.

### SUMMARY OF THE INVENTION

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The present invention relates generally to antibodies that bind, interact or otherwise associate with GM-CSF or a fragment, portion or part thereof and antagonize or neutralize GM-CSF activity. The antibodies preferably are monoclonal antibodies or antigen-binding fragments thereof. Typically, the antibodies are in isolated, homogenous or fully or partially purified form. Most typically, the antibodies are humanized or human antibodies suitable for administration to humans. These include humanized antibodies prepared, for example, from murine monoclonal antibodies, and human monoclonal antibodies which may be prepared, for example, using transgenic mice or by phage display.

In accordance with particular embodiments of the present invention humanized monoclonal antibodies are generated which bind to human GM-CSF with high affinity and which inhibit the activity of GM-CSF.

In one aspect the present invention provides a monoclonal antibody or antigen-binding fragment thereof that binds to human GM-CSF or a fragment thereof, the antibody comprising a variable light chain region comprising the sequence as set forth in SEQ ID NO:1 or a fragment or variant thereof.

In another aspect the present invention provides a monoclonal antibody or antigen-binding fragment thereof that binds to human GM-CSF or a fragment thereof, the antibody comprising a variable heavy chain sequence as set forth in SEQ ID NO:2 or a fragment or variant thereof.

In another aspect the present invention provides a monoclonal antibody or antigen-binding fragment thereof that binds to human GM-CSF or a fragment thereof, the antibody comprising a variable light chain region comprising the sequence as set forth in SEQ ID NO:1 or a fragment or variant thereof and a variable heavy chain sequence as set forth in SEQ ID NO:2 or a fragment or variant thereof.

In an embodiment, the antibody is a murine antibody or a humanized derivative thereof which inhibits the activity of GM-CSF. The antibody may be murine monoclonal antibody 4K21 deposited on 17 May 2007 at the European Collection of Cell Cultures (ECACC), Centre for Applied Microbiology and Research, Porton Down, Salisbury, United Kingdom under Accession No. 07051601.

The variable light chain region of the humanized derivative of murine monoclonal antibody 4K21 may comprise a sequence as set forth in any one of SEQ ID NOs:9 to 11 or a fragment or variant thereof. The variable heavy chain region of the humanized derivative of murine monoclonal antibody 4K21 may comprise a sequence as set forth in any one of SEQ ID NOs:13 to 15, 17 or 18 to 27, or a fragment or variant thereof.

In another aspect the invention provides a monoclonal antibody or antigen-binding fragment thereof that binds to human GM-CSF or a fragment thereof, the antibody comprising within the light chain variable region at least one complementarity determining region (CDR) comprising a sequence as set forth in any one of SEQ ID Nos:3 to 5, wherein the antibody or antigen-binding fragment thereof inhibits the activity of GM-CSF.

In an embodiment the antibody is a humanized antibody.

In another aspect the invention provides a monoclonal antibody or antigen-binding fragment thereof that binds to human GM-CSF or a fragment thereof, the antibody comprising within the heavy chain variable region at least one complementarity determining region (CDR) comprising a sequence as set forth in any one of SEQ ID Nos:6 to 8, wherein the antibody or antigen-binding fragment thereof inhibits the activity of GM-CSF.

In an embodiment the antibody is a humanized antibody.

In a further aspect the invention provides a monoclonal antibody or antigen-binding fragment thereof that binds to human GM-CSF or a fragment thereof, the antibody comprising within the light chain variable region at least one complementarity determining region (CDR) comprising a sequence as set forth in any one of SEQ ID Nos:3 to 5 and within the heavy chain variable region at least one complementarity determining region (CDR) comprising a sequence as set forth in any one of SEQ ID Nos:6 to 8.

In an embodiment the antibody is a humanized antibody. The humanized antibody may comprise a variable light chain region comprising the sequence as set forth in SEQ ID NO:9, SEQ ID NO:10 or SEQ ID NO:11 or a fragment or variant thereof. The humanized antibody may comprise a variable heavy chain region comprising the sequence as set forth in SEQ ID NO:13, SEQ ID NO:14, SEQ ID NO:15 or SEQ ID NO:17 or a fragment or variant thereof.

In an embodiment the variable heavy chain region of the humanized antibody or antigen-binding fragment thereof comprises the sequence as set forth in SEQ ID NO:13 or a fragment or variant thereof.

The variant variable heavy chain region may comprise one or more amino acid substitutions replacing amino acid residue(s) of the sequence set forth in SEQ ID NO:13, SEQ ID NO:14, SEQ ID NO:15 or SEQ ID NO:17 with amino acid residue(s) at the corresponding location(s) in the corresponding murine variable heavy chain region. In one embodiment the one or more amino acid substitutions are made to the variable heavy chain region comprising the sequence set forth in SEQ ID NO:13. In an embodiment the variant variable heavy chain region comprises a sequence as set forth in any one of SEQ ID NOs:18 to 27. In an embodiment the variant variable heavy chain region comprises the sequence as set forth in SEQ ID NO:27.

In one embodiment, the humanized antibody is selected from the group comprising the variable light chain and variable heavy chain sequences as shown in the following table:

TABLE 1

| Humanized | Heavy chain | Light chain |
|---|---|---|
| antibody | comprises | Comprises |
| hGM4/1 | SEQ ID NO: 13 | SEQ ID NO: 9 |
| hGM4/2 | SEQ ID NO: 14 | SEQ ID NO: 9 |
| hGM4/3 | SEQ ID NO: 15 | SEQ ID NO: 9 |
| hGM4/5 | SEQ ID NO: 17 | SEQ ID NO: 9 |
| hGM4/6 | SEQ ID NO: 13 | SEQ ID NO: 10 |
| hGM4/7 | SEQ ID NO: 14 | SEQ ID NO: 10 |
| hGM4/8 | SEQ ID NO: 15 | SEQ ID NO: 10 |
| hGM4/10 | SEQ ID NO: 17 | SEQ ID NO: 10 |
| hGM4/11 | SEQ ID NO: 13 | SEQ ID NO: 11 |
| hGM4/12 | SEQ ID NO: 14 | SEQ ID NO: 11 |
| hGM4/13 | SEQ ID NO: 15 | SEQ ID NO: 11 |
| hGM4/15 | SEQ ID NO: 17 | SEQ ID NO: 11 |
| hGM4/17 | SEQ ID NO: 18 | SEQ ID NO: 11 |
| hGM4/18 | SEQ ID NO: 19 | SEQ ID NO: 11 |
| hGM4/19 | SEQ ID NO: 20 | SEQ ID NO: 11 |
| hGM4/20 | SEQ ID NO: 21 | SEQ ID NO: 11 |
| hGM4/21 | SEQ ID NO: 22 | SEQ ID NO: 11 |
| hGM4/22 | SEQ ID NO: 23 | SEQ ID NO: 11 |
| hGM4/23 | SEQ ID NO: 24 | SEQ ID NO: 11 |
| hGM4/24 | SEQ ID NO: 25 | SEQ ID NO: 11 |
| hGM4/25 | SEQ ID NO: 26 | SEQ ID NO: 11 |
| hGM4/34 | SEQ ID NO: 27 | SEQ ID NO: 11 |

The invention also provides for monoclonal antibodies comprising an amino acid sequence having at least about 70% sequence identity to an amino acid sequence as set forth in any one of SEQ ID Nos: 1 to 27.

The invention also provides hybridomas producing the monoclonal antibodies of the invention.

In a further aspect the present invention provides a method for the treatment or prevention of a GM-CSF—mediated disease or condition or a disease or condition otherwise associated with elevated or aberrant GM-CSF expression and/or activity, the method comprising administering to a subject in need thereof an effective amount of at least one antibody of the invention or an antigen-binding fragment thereof.

Typically the disease or condition is an autoimmune or inflammatory disease or condition. The disease or condition may be selected from, for example, asthma, rheumatoid arthritis, chronic obstructive pulmonary disease, idiopathic thrombocytopenic purpura, acute respiratory distress syndrome, multiple sclerosis, Alzheimer's disease, Crohn's disease, irritable bowel syndrome, colitis, psoriasis, macular degeneration, uveitis, Wallerian degeneration, antiphospholipid syndrome, restinosis, atherosclerosis, idiopathic pulmonary fibrosis, relapsing polychondritis, hepatitis, glomerulonephritis, lupus and other metabolic diseases.

In a further aspect the present invention provides the use of an antibody of the invention or an antigen-binding fragment thereof for the manufacture of a medicament for treating or preventing a GM-CSF—mediated disease or condition or a disease or condition otherwise associated with elevated or aberrant GM-CSF expression and/or activity.

The invention further provides pharmaceutical compositions comprising one or more antibodies of the invention or antigen-binding fragments thereof, optionally together with suitable pharmaceutically acceptable carriers and/or diluents.

### BRIEF DESCRIPTION OF THE DRAWINGS

The present invention will now be described, by way of non-limiting example only, with reference to the accompanying drawings.

FIG. 1. The ability of 13 murine anti-GM-CSF monoclonal antibodies to neutralize the cell growth promoting activity of human GM-CSF, as compared to a commercially available anti-GM-CSF antibody (BD).

FIG. 2. Proliferation of murine FDC-P1 cells in the presence of murine anti-GM-CSF monoclonal antibodies, as compared to a commercially available anti-GM-CSF antibody (BD).

FIG. 3. Alignment of variable light chain sequences of 4K21 monoclonal antibody and four humanized variants. CDRs L1, L2 and L3 are indicated. Mutations in framework 4, G105Q and V109L, are also shown.

FIG. 4. Alignment of variable heavy chain sequences of 4K21 monoclonal antibody and five humanized variants. CDRs H1, H2 and H3 are indicated.

FIG. 5. Binding affinities (RU) of 15 humanized 4K21 monoclonal antibodies for human GM-CSF as determined by BIAcore analysis, compared to murine 4K21 monoclonal antibody and a commercially available anti-GM-CSF antibody (BD). For simplicity, every second humanized monoclonal antibody is numbered (4K21-1, 4K21-3, etc).

FIG. 6. BIACore binding analysis of humanized 4K21 antibodies. Antibodies were injected at concentrations from 66.7 nM to 4.17 nM with two-fold serial dilutions. The binding affinity (A), association rate (B) and dissociation rate (C) of the GM-CSF humanized antibodies are shown.

FIG. 7. Alignment of variable heavy chain sequences of 4K21 monoclonal antibody and five humanized variants showing location and identity (boxes) of eight amino acid residue differences between 4K21 murine framework and humanised h4Vh/10 framework. Numbers (19-25) above boxed residues indicate identities of humanised antibodies constructed with back mutations. CDRs H1, H2 and H3 are also indicated.

FIG. 8. Humanized antibodies neutralize the cell growth promoting activity of human GM-CSF. Antibodies were prepared at concentrations from 400 nM to 28 fM with three-fold serial dilution and mixed with GM-CSF for 1 hr before addition of cells. Cells were allowed to grow for 72 hrs and then cell growth was quantitated by incorporation of ³H-thymidine. Each point was calculated in triplicate.

FIG. 9. BIACore binding analysis of 4K21 and humanized 4K21 antibodies hGM4/11, hGM4/21, hGM4/22 and hGM4/34. Antibodies were coupled to a CM5 chip via an anti murine IgG or anti human IgG antibody. GM-CSF was injected at concentrations of 1000 nM to 15.63 nM with 2-fold serial dilution. The binding affinity (A), association rate (B) and dissociation rate (C) of the GM-CSF antibodies are shown.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A sequence listing is provided at the end of the specification. Specifically, the amino acid sequences of variable light chains and variable heavy chains and CDRs of antibodies in accordance with the invention are set forth in SEQ ID Nos:1 to 27.

### DETAILED DESCRIPTION OF THE INVENTION

The articles “a” and “an” are used herein to refer to one or to more than one (i.e., to at least one) of the grammatical object of the article. By way of example, “an element” means one element or more than one element.

The term “polypeptide” means a polymer made up of amino acids linked together by peptide bonds. The terms “polypeptide” and “protein” are used interchangeably herein, although for the purposes of the present invention a “polypeptide” may constitute a portion of a full length protein.

In the context of the present specification reference to an “antibody” or “antibodies” includes reference to all the various forms of antibodies, including but not limited to whole antibodies, antibody fragments, including, for example, Fv, Fab, Fab′ and F(ab′)₂ fragments, humanized antibodies, human antibodies and immunoglobulin-derived polypeptides produced through genetic engineering techniques.

In the context of the present specification reference to “binding” of an antibody means binding, interacting or associating with or to a target antigen such as GM-CSF. Reference to “GM-CSF” includes fragments or portions thereof which comprise the epitopes to which an antibody binds. Consequently, reference to an antibody binding to GM-CSF includes within its scope the binding, interaction or association of the antibody or an antigen-binding portion thereof to part, fragment or epitope-containing region of GM-CSF. Generally, “binding”, “interaction” or “association” means or includes the specific binding, interaction or association of the antibody to GM-CSF or a portion thereof.

The terms “inhibits” and “inhibiting” as used herein as they relate to the activity of GM-CSF does not necessarily mean completely inhibiting activity. Rather, activity may be inhibited to an extent, and/or for a time, sufficient to produce the desired effect. Thus inhibition of GM-CSF activity may be partial or complete attenuation of one or more biological effects of GM-CSF and such inhibition may be temporally and/or spatially limited. By temporally and/or spatially limited is meant that the inhibition may be limited to particular physiological conditions or circumstances and/or to particular regions of the body.

In the context of this specification, the term “activity” as it relates to GM-CSF means any cellular function, action, effect or influence exerted by the GM-CSF, either by the protein or polypeptide itself or any fragment or portion thereof.

As used herein the terms “treating”, “treatment”, “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus the terms “treating” and “preventing” and the like are to be considered in their broadest context. For example, treatment does not necessarily imply that a patient is treated until total recovery.

As used herein the term “effective amount” includes within its meaning a non-toxic but sufficient amount of an agent to provide the desired effect. The exact amount or dose required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

The term “subject” as used herein typically refers to mammals including humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal. Even more preferably, the mammal is a human.

The present invention provides antibodies that function as GM-CSF antagonists and may be used for treating certain conditions induced by or otherwise associated with elevated levels and/or activity of GM-CSF. The present invention also provides methods for treating these conditions comprising administering an anti-GM-CSF antibody of the invention to a patient afflicted with such a condition. Also provided are compositions for use in such methods, the compositions comprising one or more anti-GM-CSF antibodies.

The antibodies of the present invention bind, interact or otherwise associate with GM-CSF or a portion thereof. The antibodies are typically specific for GM-CSF from a particular species, such as human GM-CSF, or, in view of the level of sequence similarity between GM-CSF from different species, the antibodies may show some cross-reactivity with GM-CSF from two or more species. In the case of antibodies directed towards human GM-CSF, some level of cross-reactivity with other mammalian forms of GM-CSF may be desirable in certain circumstances, such as for example, for testing antibodies in animal models of a particular disease and for conducting toxicology, safety and dosage studies.

Typically, antibodies of the invention are monoclonal antibodies or antigen-binding fragments thereof. Most preferably, the antibodies are humanized or human antibodies suitable for administration to humans. These include humanized antibodies prepared, for example, from murine monoclonal antibodies and human monoclonal antibodies which may be prepared, for example, using transgenic mice or by phage display.

Antibodies of the present invention may be prepared by a variety of procedures well known to those skilled in the art. For example, reference may be had to Monoclonal Antibodies, Hybridomas: A New Dimension in Biological Analyses, Kennet et al. (eds.), Plenum Press, New York (1980); Antibodies: A Laboratory Manual, Harlow and Land (eds.), Cold Spring Harbor Laboratory Press, Cold Spring Harbor, N.Y., (1988); and Monoclonal Antibodies: Principles and Practice, Goding, 3rd Edition, Academic Press (1996). The disclosures thereof are incorporated herein by reference in their entirety. Similarly, monoclonal antibodies secreted by hybridoma cell lines may be purified by conventional techniques.

By way of example, one method for producing an antibody of the present invention comprises immunizing a non-human animal, such as a mouse or a transgenic mouse, with a GM-CSF polypeptide, or immunogenic portion or fragment thereof, whereby antibodies directed against the GM-CSF polypeptide are generated in said animal. The GM-CSF polypeptide or immunogenic portion or fragment thereof that may be used to immunize animals may be from any mammalian source. Typically, the GM-CSF polypeptide or immunogenic portion of fragment thereof is GM-CSF.

Antigen-binding fragments of antibodies of the present invention may be produced by conventional techniques. Examples of such fragments include, but are not limited to, Fab, Fab′, F(ab′) 2 and Fv fragments, including single chain Fv fragments (termed sFv or scFv). Antibody fragments and derivatives produced by genetic engineering techniques, such as disulphide stabilized Fv fragments (dsFv), single chain variable region domain (Abs) molecules and minibodies are also contemplated for use. Unless otherwise specified, the terms “antibody” and “monoclonal antibody” as used herein encompass both whole antibodies and antigen-binding fragments thereof.

Such derivatives of monoclonal antibodies directed against GM-CSF may be prepared and screened for desired properties, by known techniques. The techniques may involve, for example, isolating DNA encoding a polypeptide chain (or a portion thereof) of a monoclonal antibody of interest, and manipulating the DNA through recombinant DNA technology. The DNA may be used to generate another DNA of interest, or altered (e.g. by mutagenesis or other conventional techniques) to add, delete, or substitute one or more amino acid residues, for example. DNA encoding antibody polypeptides (e.g. heavy or light chain, variable region only or full length) may be isolated from B-cells of mice that have been immunized with GM-CSF. The DNA may be isolated by conventional procedures including polymerase chain reaction (PCR).

Phage display is an alternative example of a suitable technique whereby derivatives of antibodies of the invention may be prepared. In one approach, polypeptides that are components of an antibody of interest are expressed in any suitable recombinant expression system, and the expressed polypeptides are allowed to assemble to form antibody molecules.

Single chain antibodies may be formed by linking heavy and light chain variable region (Fv region) fragments via an amino acid bridge (short peptide linker), resulting in a single polypeptide chain. Such single-chain Fvs (scFvs) may be prepared by fusing DNA encoding a peptide linker between DNAs encoding the two variable region polypeptides (VL and VH). The resulting antibody fragments can form dimers or trimers, depending on the length of a flexible linker between the two variable domains (see Kortt et al., Protein Engineering 10: 423, 1997). Techniques developed for the production of single chain antibodies include those described in U.S. Pat. No. 4,946,778; Bird (Science 242: 423, 1988), Huston et al. (Proc. Natl. Acad Sci USA 85: 5879, 1988) and Ward et al. (Nature 334: 544, 1989). The disclosures thereof are incorporated herein by reference in their entirety. Single chain antibodies derived from antibodies provided herein are encompassed by the present invention.

An example of a monoclonal antibody contemplated by the present invention is murine monoclonal antibody 4K21, the generation of which is described herein. Murine monoclonal antibody 4K21 comprises the variable light chain sequence as set forth in SEQ ID NO:1 and the variable heavy chain sequence as set forth in SEQ ID NO:2. The sequences of the light chain CDRs of 4K21 are set forth in SEQ ID Nos:3 to 5. The sequences of the heavy chain CDRs of 4K21 are set forth in SEQ ID Nos:6 to 8.

A hybridoma producing murine monoclonal antibody 4K21 was deposited on 17 May 2007 at the European Collection of Cell Cultures (ECACC), Centre for Applied Microbiology and Research, Porton Down, Salisbury, United Kingdom, under Accession No. 07051601.

It will be appreciated that the amino acid sequences of monoclonal antibodies of the invention may include one or more amino acid substitutions such that although the primary sequence of the polypeptide is altered, the ability of the antibody to bind GM-CSF and the activity of the antibody is retained. The substitution may be a conservative substitution. The term “conservative amino acid substitution” as used herein refers to a substitution or replacement of one amino acid for another amino acid with similar properties within a polypeptide chain (primary sequence of a protein). For example, the substitution of the charged amino acid glutamic acid (Glu) for the similarly charged amino acid aspartic acid (Asp) would be a conservative amino acid substitution.

The present invention contemplates variants of the light chain and heavy chain sequences disclosed herein and such variants are encompassed within the scope of the present invention. The term “variant” as used herein refers to substantially similar sequences. Generally, polypeptide sequence variants possess qualitative biological activity in common. A variant polypeptide sequence may be a derivative of a sequence as disclosed herein, which derivative comprises the addition, deletion, or substitution of one or more amino acids. Variants may differ from the disclosed sequences within framework regions or within CDRs of either the light or heavy chain sequences. For example, monoclonal antibodies or antigen-binding fragments thereof comprising amino acid sequences having at least about 70% sequence identity to the amino acid sequences set forth in SEQ ID Nos: 1 to 27 are contemplated. The monoclonal antibody or antigen-binding fragment thereof may comprise amino acid sequences having at least about 75%, 80%, 85%, 90%, 95%, 96%, 97%, 98% or 99% sequence identity to the amino acid sequences set forth in SEQ ID Nos: 1 to 27. The term “variant” encompasses antibody sequences modified from those disclosed herein by any suitable means. For example, when used in the context of murine sequences, the term “variant” includes within its scope humanized forms of such sequences. When used in the context of humanized sequences disclosed herein the term “variant” includes within its scope modified sequences comprising one or more murine back mutations.

Antibodies derived from non-human animals, for example mice, are generally unsuitable for administration to humans as they may cause an immune response and result in the generation of anti-mouse antibodies (the so-called HAMA response). The HAMA response can neutralize the mouse antibodies by rapidly clearing them from the blood, thus preventing the mouse antibody from binding to its target.

To avoid development of a HAMA response one strategy is to “humanize” the mouse antibody by replacing as many “foreign” residues in the non-epitope binding regions with human sequences. The specificity of the interaction between an antibody and an antigen involves the hypervariable or complementarity-determining regions (CDRs) in the variable domain. These residues are generally not changed during the humanization process. The remaining residues in the variable domain, referred to as the framework (FW) and the constant regions of the antibody, on both heavy and light chains are usually replaced with human sequences. To avoid disrupting the structure of the antibody-binding pocket, and the specificity or affinity of the antibody, certain mouse residues in the framework regions may need to be preserved. Suitable humanization processes, such as CDR grafting, are well known to those skilled in the art. A particularly suitable approach is exemplified herein. Procedures for the production of chimeric and humanized monoclonal antibodies also include those described in, for example, Riechmann et al., Nature 332: 323, 1988, Liu et al., Proc. Natl. Acad. Sci. USA 84: 3439, 1987, Larrick et al., Bio/Technology 7: 934, 1989 and Winter and Harris, TIPS 14: 139, 1993. The complementarity determining regions (CDRs) of a given antibody may be identified using the system described by Kabat et al. in Sequences of Proteins of Immunological Interest, 5th Ed., US Dept. of Health and Human Services, PHS, NIH, NIH Publication No. 91-3242, 1991).

For example, as described herein the murine monoclonal antibody 4K21 has been subjected to humanization to reduce the immunogenicity of the antibody. Twenty two humanized antibodies retaining high binding affinity (picomolar range) for human GM-CSF and retaining suitable GM-CSF inhibitory activity were identified, as exemplified herein.

In particular embodiments, humanized antibodies of the invention comprise, within the variable region of their light chain, at least one of the CDRs found in the light chain of murine antibody 4K21, as set forth in SEQ ID Nos:3 to 5. Thus, among the antibodies contemplated by the present invention are those that comprise from one to all three of the CDR sequences from the light chain variable region of murine antibody 4K21.

Further, among the antibodies contemplated by the present invention are those that comprise, within the variable region of their heavy chain, at least one of the CDRs found in the heavy chain of murine antibody 4K21, as set forth in SEQ ID Nos:6 to 8. Thus, among the antibodies contemplated by the present invention are those that comprise from one to all three of the CDR sequences from the heavy chain variable region of murine antibody 4K21.

In a preferred embodiment, the antibodies of the present invention comprise from one to all six CDR sequences from the heavy and light chain variable regions of murine antibody 4K21.

Humanized antibodies in accordance with particular embodiments of the invention are described in Table 2 below.

TABLE 2
Humanized antibodies derived from
murine monoclonal antibody 4K21

| Humanized | Heavy chain | Light chain |
|---|---|---|
| antibody | comprises | comprises |
| hGM4/1 | SEQ ID NO: 13 | SEQ ID NO: 9 |
| hGM4/2 | SEQ ID NO: 14 | SEQ ID NO: 9 |
| hGM4/3 | SEQ ID NO: 15 | SEQ ID NO: 9 |
| hGM4/4 | SEQ ID NO: 16 | SEQ ID NO: 9 |
| hGM4/5 | SEQ ID NO: 17 | SEQ ID NO: 9 |
| hGM4/6 | SEQ ID NO: 13 | SEQ ID NO: 10 |
| hGM4/7 | SEQ ID NO: 14 | SEQ ID NO: 10 |
| hGM4/8 | SEQ ID NO: 15 | SEQ ID NO: 10 |
| hGM4/9 | SEQ ID NO: 16 | SEQ ID NO: 10 |
| hGM4/10 | SEQ ID NO: 17 | SEQ ID NO: 10 |
| hGM4/11 | SEQ ID NO: 13 | SEQ ID NO: 11 |
| hGM4/12 | SEQ ID NO: 14 | SEQ ID NO: 11 |
| hGM4/13 | SEQ ID NO: 15 | SEQ ID NO: 11 |
| hGM4/14 | SEQ ID NO: 16 | SEQ ID NO: 11 |
| hGM4/15 | SEQ ID NO: 17 | SEQ ID NO: 11 |
| hGM4/17 | SEQ ID NO: 18 | SEQ ID NO: 11 |
| hGM4/18 | SEQ ID NO: 19 | SEQ ID NO: 11 |
| hGM4/19 | SEQ ID NO: 20 | SEQ ID NO: 11 |
| hGM4/20 | SEQ ID NO: 21 | SEQ ID NO: 11 |
| hGM4/21 | SEQ ID NO: 22 | SEQ ID NO: 11 |
| hGM4/22 | SEQ ID NO: 23 | SEQ ID NO: 11 |
| hGM4/23 | SEQ ID NO: 24 | SEQ ID NO: 11 |
| hGM4/24 | SEQ ID NO: 25 | SEQ ID NO: 11 |
| hGM4/25 | SEQ ID NO: 26 | SEQ ID NO: 11 |
| hGM4/34 | SEQ ID NO: 27 | SEQ ID NO: 11 |

Procedures for generating human antibodies in non-human animals have also been developed and are well known to those skilled in the art. The antibodies may be partially human, or completely human. For example, transgenic mice into which genetic material encoding one or more human immunoglobulin chains has been introduced may be used to produce antibodies. The transgenic mice may be such that human immunoglobulin polypeptide chains replacing endogenous immunoglobulin chains are present in at least some antibodies produced by the animal upon immunization.

Another method for generating human antibodies is phage display. Phage display techniques for generating human antibodies are well known to those skilled in the art, and include the methods used by Cambridge Antibody Technology and MorphoSys and which are described in International Patent Publication Nos. WO 92/01047, WO 92/20791, WO 93/06213 and WO 93/11236 (the disclosures of which are incorporated herein by reference).

Antibodies of the present invention or hybridomas comprising such antibodies may be screened and manipulated further to identify monoclonal antibodies with particularly desirable properties, such as increased binding affinity, reduced immunogenicity and/or increased inhibitory activity against GM-CSF.

The present invention provides methods for treating or preventing GM-CSF— mediated diseases or conditions, diseases or conditions otherwise associated with elevated levels and/or activity of GM-CSF, and other diseases or conditions which may be beneficially treated by inhibiting or neutralizing GM-CSF activity by the administration of antibodies of the present invention. Diseases and conditions which may be treated in accordance with the present invention include autoimmune and inflammatory diseases. Such diseases include but are not limited to asthma, rheumatoid arthritis, chronic obstructive pulmonary disease, idiopathic thrombocytopenic purpura, acute respiratory distress syndrome, multiple sclerosis, Alzheimer's disease, Crohn's disease, irritable bowel syndrome, colitis, psoriasis, macular degeneration, uveitis, Wallerian degeneration, antiphospholipid syndrome, restinosis, atherosclerosis, idiopathic pulmonary fibrosis, relapsing polychondritis, hepatitis, glomerulonephritis, lupus and other metabolic diseases. Additional autoimmune diseases which may be treated in accordance with the invention include systemic sclerosis, scleroderma, Sjogren syndrome, spondyloarthritis, Sapho syndrome, juvenile iodipathic arthritis, lyme disease, polymyositis, dermatomyositis, autoimmune thyroditis, Grave's disease, Type 1 diabetes, adrenaltis, autoimmune Addison's disease, polyendocrine syndromes, gastritis, pernicious anemia, hypophysitis, hemolytic anemia, neutropenia, aplastic anemia, clotting disorder including acquired von Willebrand syndrome, Guillain-Barre Syndrome, chronic inflammatory demyelinating polyradiculoneuropathy, myasthenia gravis, Lambert-Eaton myasthenic syndrome, acquired neuromyotonia, Stiff-Person Syndrome, Cerebellar Ataxia, Rasmussen Encephalitis, Morvan Syndrome, Limbic encephalitis, ocular disease, inner ear disease, celiac disease, primary billary cirrhosis, primary sclerosing cholangitis, pancreatitis, pemphigus pemphigoid, alopecia greata, vitiligo, chronic urticaria, Goodpasture's disease, ANCA-associated glomerulonephritis, orchitis, oophoritis, rheumatic heart disease, myocarditis, dilated cardiomyopathy, polyartheritis nodosa, Kawasaki's disease, Wegener's granulomatosis, microscopic polyangiitis, Churg-Strauss syndrome, cryoglobulenemic vasculitis, Henoch-Schonlein purpura, Behcet's disease, giant cell arteritis, Takayasu's arteritis, idiopathic bronchiolitis obliterans, idiopathic pulmonary fibrosis, autoimmune disorder of the lung and Opsoclonus-Myoclonus syndrome.

Antibodies disclosed herein also find application in the treatment of failed or rejected implants and prostheses and failed or rejected organ transplants, such as for example lung, kidney, heart and liver.

Additional applications, both in vivo and in vitro, of antibodies of the invention are contemplated. For example, antibodies of the invention may be employed in assays designed to detect the presence of GM-CSF and/or to purify GM-CSF. Antibodies may also be tested in animal models of particular diseases and for conducting toxicology, safety and dosage studies.

For therapeutic and prophylactic applications, antibodies of the invention are administered to a subject in need thereof in an amount effective to obtain the desired therapeutic or prophylactic effect. It will be understood that the specific effective amount or dose for any particular subject will depend upon a variety of factors including, for example, the activity of the specific antibody(ies) employed, the age, body weight, general health and diet of the individual to be treated, the time of administration, rate of excretion, and combination with any other treatment or therapy. Single or multiple administrations can be carried out with dose levels and pattern being selected by the treating physician.

In treating or preventing autoimmune and inflammatory conditions, the present invention contemplates the administration of multiple antibodies if required or desirable. Whether it is suitable or desirable to administer one or more antibodies can be determined by those skilled in the art on a case-by-case basis.

The invention also contemplates combination therapies, wherein antibodies as described herein are coadministered with other suitable agents which may facilitate the desired therapeutic or prophylactic outcome. For example, in the context of asthma, one may seek to maintain ongoing anti-inflammatory therapies in order to control the incidence of inflammation whilst employing agents in accordance with embodiments of the present invention. By “coadministered” is meant simultaneous administration in the same formulation or in two different formulations via the same or different routes or sequential administration by the same or different routes. By “sequential” administration is meant a time difference of from seconds, minutes, hours, days, weeks, months or years between the administration of the two agents. These agents may be administered in any order.

According to embodiments of the invention, antibodies may be administered in any suitable form. In accordance with the present invention antibodies are typically administered in the form of pharmaceutical compositions, which compositions may comprise one or more pharmaceutically acceptable carriers, excipients or diluents. Such compositions may be administered systemically, regionally or locally and via any suitable route such as by parenteral (including intravenous, intraarterial or intramuscular), oral, nasal, topical and subcutaneous routes.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration. Adjuvants typically include emollients, emulsifiers, thickening agents, preservatives, bactericides and buffering agents.

For administration as an injectable solution or suspension, non-toxic parenterally acceptable diluents or carriers can include, Ringer's solution, medium chain triglyceride (MCT), isotonic saline, phosphate buffered saline, ethanol and 1,2 propylene glycol. Methods for preparing parenterally administrable compositions are known to those skilled in the art, and are described in more detail in, for example, Remington's Pharmaceutical Science, 15th ed., Mack Publishing Company, Easton, Pa., hereby incorporated by reference in its entirety.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in any country in the field of endeavour to which this specification relates.

The present invention will now be described with reference to the following specific examples, which should not be construed as in any way limiting the scope of the invention.

### EXAMPLES

### Example 1

### Generation of Murine Monoclonal Antibody 4K21

Anti-human GM-CSF antibodies were generated by intraperitoneal injection of 50 μg of recombinant human GM-CSF (Peprotech) in CFA (complete Freund's adjuvant) into BALB/c mice. This was followed by two further injections of 25 μg of recombinant human GM-CSF in IFA (incomplete Freund's adjuvant) 2 weeks and 4 weeks later.

Six months after the initial injection, a 25 μg booster of recombinant human GM-CSF in IFA was administered intraperitoneally. Finally 2 weeks after the booster 50 μg of recombinant human GM-CSF in phosphate buffered saline was administered intravenously. Five days later mice spleens were harvested and fused with SP2/0 cells.

Antibody expressing hybridoma supernatants were screened by ELISA (enzyme linked immunosorbent assay) for specific binding to recombinant human GM-CSF. Anti-human GM-CSF antibodies were further screened for neutralizing activity with the TF-1 and the FDC-P1 bioassays (see Example 2).

One such murine anti-human GM-CSF monoclonal antibody generated was designated 4K21-O11-E14 (hereinafter 4K21).

The variable region amino acid sequences of mAb 4K21 are as follows:

### Light Chain

(SEQ ID NO: 1)
DVVMTQTPLSLPVSLGDQASISCRSSQSLVNSNGNTYLHWFLQKPGQS
PKLLIYKVSNRFSGVPDRFSGSGSGTDFTLKISRVEAEDLGVYFCSQS
THVPPTFGGGTKLEIK

### Heavy Chain

(SEQ ID NO: 2)
EVQLVESGGGLVKSGGSLKLSCAASGFAFSAYDMSWVRQTPEKRLELV
AYISSGGSSFYYPDTVKGRFTISRDNAKNTLYLQMSSLKSEDTAMYYC
TRHLGFDYWGQGTTLTVSS

The hybridoma containing 4K21 was deposited on 17 May 2007 at the European Collection of Cell Cultures (ECACC), Centre for Applied Microbiology and Research, Porton Down, Salisbury, United Kingdom, under Accession No. 07051601.

### Example 2

### Biological Properties of Murine Monoclonal Antibody 4K21

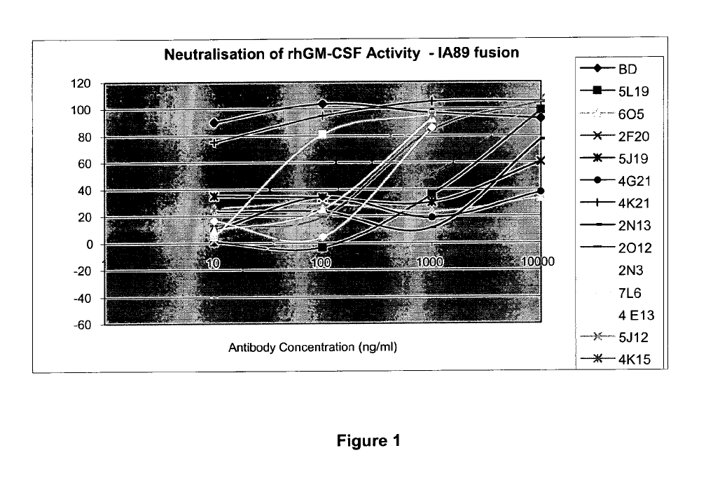

Murine anti-human GM-CSF monoclonal antibody 4K21 was demonstrated to be able to neutralize the growth promoting function of human GM-CSF in a TF-1 cell growth bioassay (FIG. 1). TF-1 cells are dependant on the presence of GM-CSF for their growth and are maintained with 2 ng/ml recombinant GM-CSF (Peprotech). To perform the neutralization assay, a range of antibody concentrations (10 ng/ml to 10,000 ng/ml) was prepared in TF-1 growth media with 0.25 ng/ml GM-CSF. The antibody and GM-CSF were mixed at each concentration and incubated for 1 hr at 37° C. in a 96 well plate. Washed TF-1 cells (1×10⁵) were then added to each well and incubated at 37° C. for 72 hrs. Cellular growth was quantitated by pulsing each well for 4 hrs with 0.5 uCi of ³H-thymidine.

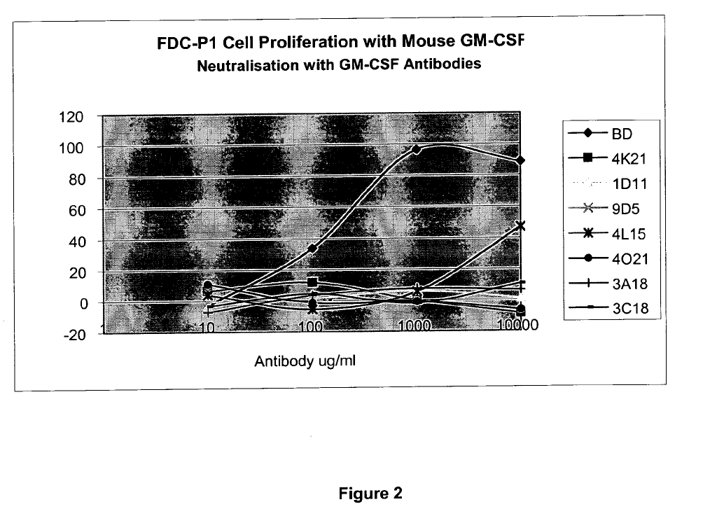

In contrast, demonstrating the anti-human GM-CSF specificity of 4K21, this antibody was not able to neutralize murine GM-CSF mediated growth of FDC-P1 cells (FIG. 2). To perform the neutralization assay, a range of antibody concentrations (10 ng/ml to 10,000 ng/ml) was prepared in FDC-P1 growth media with 5 ng/ml murine GM-CSF. The antibody and GM-CSF were mixed at each concentration and incubated for 1 hr at 37° C. in a 96 well plate. Washed FDC-P1 cells (1×10⁵) were then added to each well and incubated at 37° C. for 72 hrs. Cellular growth was quantitated by pulsing each well for 4 hrs with 0.5 uCi of ³H-thymidine.

The binding kinetics of the 4K21 monoclonal antibody was determined using a Biacore and compared with the binding kinetics for a commercially available rat anti-human GM-CSF antibody from BD Biosciences (BD; Cat No. 554501) (see Table 1 below). Recombinant human GM-CSF was coated onto a CM5 chip to determine the binding kinetics of the 4K21 antibody. An uncoated flow cell was used as a reference. The antibody, prepared in HBS-EP at concentrations of 66.7 nM to 4.17 nM with 2-fold serial dilution, was injected for 2 minutes with a stabilisation time of 10 minutes and dissociation time of 15 minutes. The BIACore kinetic analysis wizard was run to determine each antibody's association (ka [1/Ms]), dissociation (kd[1/s]) and affinity (KD [nM]) to GM-CSF. As shown in Table 3, 4K21 displays an extremely high binding affinity for human GM-CSF, binding at low picomolar concentrations.

TABLE 3
Binding kinetics of 4K21 monoclonal antibody.

| Antibody | ka (1/Ms) | kd (1/s) | KA (1/M) | KD (M) | KD (nm) |
|---|---|---|---|---|---|
| BD | 5.20E+04 | <1e−5 | >5.2e9 | <1.9e−10 | <0.19 |
| 4K21-O11-E14 | 5.90E+04 | <1e−5 | >5.9e9 | <1.7e−10 | <0.17 |
| (mouse) | | | | | |

Recombinant human GM-CSF was coated onto a CM5 chip to determine the binding kinetics of the 4K21 antibody.

### Example 3

### Humanization of 4K21

### Defining CDR and Framework Residues.

The CDR and framework regions of an antibody are usually defined according to various numbering schemes such as Kabat, Chothia or IMGT (ImMunoGeneTics information System® http://imgt.cines.fr). The Kabat definition is based on sequence variability and is the most commonly used. However, the CDRs for a given antibody as defined by Kabat are not necessarily identical to the CDRs defined by the other numbering systems. The CDRs defined by two numbering systems may overlap, or one may extend a few residues either side of the other.

A combination of the Kabat and IMGT numbering systems was used to define the CDRs and framework regions in the variable (V) domain. The aim was to maximise the extent of the mouse CDR sequences that were grafted into the human framework in order to preserve the structure of the antigen-binding pocket. Accordingly, the GM-CSF antibody CDRs included all residues classified as CDR by both the Kabat and IMGT numbering systems. The remaining sequences comprised the V domain framework.

### Selecting Suitable Human Antibody Framework Sequences

To select suitable human antibody framework sequences onto which the mouse CDRs were grafted a number of strategies were used:
Sequence databases were searched using BlastP and identified human Ig V region light and heavy chain sequences with the highest homology to mouse variable regions of the GM-CSF antibody.
The human IgV region heavy and light chain sequences were ranked based on three criteria:
   a) overall similarity and identity to complete murine variable region sequence
   b) canonical structure comparison
   c) framework similarity score
Known human antibodies with the highest homology to the mouse GM-CSF antibody were selected and those human variable region frameworks were used to graft the mouse GM-CSF antibody CDRs.
   Selecting Homologous Antibodies from Sequence Databases

The mouse 4K21 variable region amino acid sequences (both heavy and light chain) were individually used as the query sequence in BlastP searches of the NCBI non-redundant databases. Sequences derived from human sources were then selected. The reference was checked and in instances in which it was not clear that the clone was truly isolated from human sources the sequences were ignored. The light chain canonical structure was determined based on the definition in Tomlinson et al., EMBO J. 14:4628-4638, 1995. Similarly, the heavy chain canonical structure was determined base on the definition in Chothia et al., J. Mol. Biol. 227:799-817, 1992.

A list of sequences with the highest homology to the query sequence was generated from each search and is tabulated below in Tables 4 and 5.

Selection of human light chain framework

TABLE 4
Human sequences homologous to the mouse GM-CSF mAb variable light chain.

| | | | | | Framework | |
|---|---|---|---|---|---|---|
| hIgLV | | | | Cannonical | similarity | |
| GMCSF | Sequence ID | % Identity | % similarity | structure | score | reference |
| 1 | 1X9Q_A | 93 | 3 | 4-1-1 | | Probably murine |
| | GI:62738392 | | | | | |
| 2 | BAC01734.1 | 83 | 8 | 4-1-1 | −11 | no reference: |
| | GI:21669419 | | | | | human |
| | | | | | | therapeutic |
| | | | | | | library clone |
| 3 | 1WT5_D | 79 | 11 | | | |
| | GI:67464418 | | | | | |
| 4 | ABC66847.1 | 82 | 8 | 4-1-1 | | Probably murine |
| | GI:84797796 | | | | | |
| 5 | BAC01730.1 | 83 | 7 | 4-1-1 | −11 | no reference: |
| | GI:21669411 | | | | | human |
| | | | | | | therapeutic |
| | | | | | | library clone |
| 6 | AAZ09071.1 | 83 | 6 | 4-1-3 | −11 | ref - human |
| | GI:70798723 | | | | | antibody |
| 7 | ABC66863.1 | 82 | 8 | 4-1-1 | | Probably murine |
| | GI:84797828 | | | | | |
| 8 | ABC66914.1 | 78 | 11 | 4-1-1 | | Probably murine |
| | GI:84797930 | | | | | |
| 9 | BAC01562.1 | 78 | 11 | 4-1-1 | −10 | no reference: |
| | GI:21669075 | | | | | human |
| | | | | | | therapeutic |
| | | | | | | library clone |
| 10 | AAK94811.1 | 77 | 12 | | | |
| | GI:18025604 | | | | | |
| 11 | CAA51134.1 | 76 | 12 | | | |
| | GI:441401 | | | | | |
| 12 | CAA61441.1 | 76 | 12 | | | |
| | GI:929641 | | | | | |
| 13 | CAB51297.1 | 77 | 12 | | | |
| | GI:5578794 | | | | | |
| 14 | CAB51293.1 | 78 | 12 | | | |
| | GI:5578786 | | | | | |
| 15 | ABC66930.1 | 78 | 9 | 4-1-1 | | Probably murine |
| | GI:84797962 | | | | | |
| 16 | ABC66929.1 | 78 | 9 | | | |
| | GI:84797960 | | | | | |
| 17 | ABA26215.1 | 77 | 11 | | | |
| | GI:75707503 | | | | | |
| 18 | ABA71374.1 | 74 | 11 | | | |
| | GI:77379434 | | | | | |
| 19 | AAY33347.1 | 78 | 10 | 4-1-1 | −10 | ref - human |
| | GI:63102883 | | | | | antibody |
| 20 | ABA70842.1 | 75 | 12 | | | |
| | GI:77378240 | | | | | |
| 21 | ABA26227.1 | 76 | 11 | | | |
| | GI:75707527 | | | | | |
| 22 | BAC01733.1 | 78 | 10 | | | |
| | GI:21669417 | | | | | |
| 23 | ABC66952.1 | 76 | 10 | | | |
| | GI:84798006 | | | | | |
| 24 | ABA260421 | 75 | 12 | | | |
| | GI:75707157 | | | | | |
| 25 | AAY33405.1 | 78 | 11 | | | |
| | GI:63102999 | | | | | |
| 26 | ABC66931.1 | 76 | 11 | | | |
| | GI:84797964 | | | | | |
| 27 | AAB61659.1 | 76 | 11 | | | |
| | GI:2072982 | | | | | |
| 28 | AAM46359.1 | 72 | 6 | | | |
| | GI:21310712 | | | | | |
| 29 | 1S3K_L | 66 | 15 | | | |
| | GI:51247325 | | | | | |

The frameworks with the highest homology to the 4K21 light chain variable regions were hIgVL GMCSF01, 02, 04, 05, 06, 07, 19, 15, 09 and 08. All these antibodies have the same canonical structure as 4K21 light chain (4-1-1) except for hIgVL GMCSF06 (4-1-3). The source of a number of these antibodies was not clear and so they were ignored.

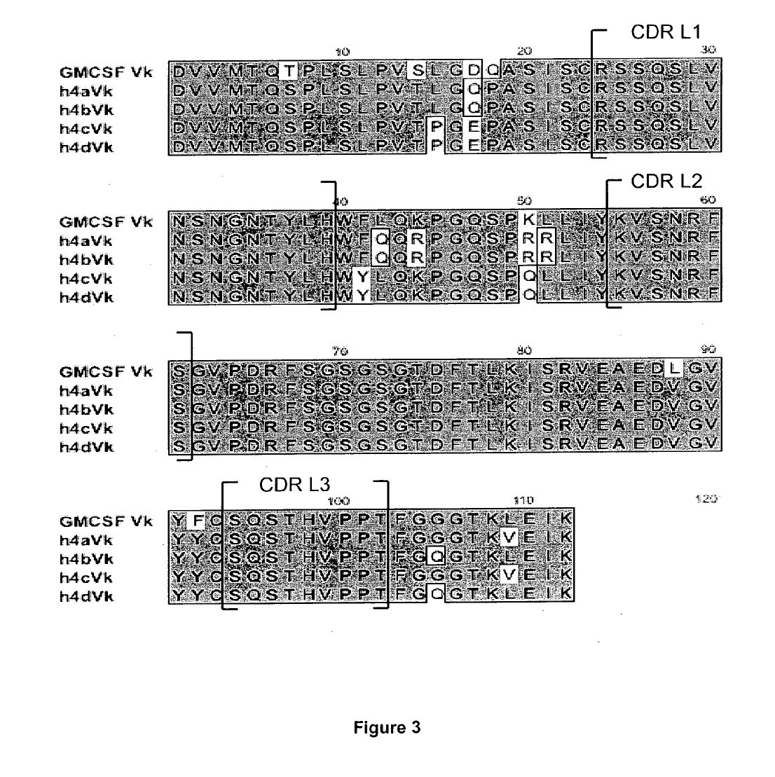

The antibodies selected for comparison were hIgVL GMCSF02, 05, 06, 19 and 09. Antibodies O₂ and 05 have the same framework so a total of 4 different frameworks were defined. The frameworks defined are quite similar, and in fact, two pairs of frameworks are identical except for two mutations in framework 4. The mutations are the same in both pairs—G105Q and V109L—see FIG. 3. Based on this data it was decided to synthesize only h4a, h4b and h4c as the mutations in framework 4 would be covered by this combination of light chains if they were important.

### Selection of Human Heavy Chain Framework

TABLE 5
Human sequences homologous to the mouse GM-CSF mAb variable heavy chain.

| | | | | | Framework | |
|---|---|---|---|---|---|---|
| hIgHV | | | | Canonical | similarity | |
| GMCSF | Sequence ID | % Identity | % similarity | structure | score | reference |
| 1 | AAX82494.1 | 80 | 6 | 1-3 | −8 | ref - human |
| | GI:62421461 | | | | | antibody |
| 2 | 1FH5_H | 80 | 6 | 1-3 | −9 | Probably |
| | GI:10835839 | | | | | murine |
| 3 | AAC51009.1 | 72 | 12 | | −17 | |
| | GI:1791031 | | | | | |
| 4 | CAC28887.1 | 72 | 13 | | −18 | |
| | GI:12733968 | | | | | |
| 5 | BAC01301.1 | 74 | 12 | 1-3 | −16 | no reference - |
| | GI:21668548 | | | | | human |
| | | | | | | therapeutic |
| | | | | | | library clone |
| 6 | S22657 | 71 | 13 | | −17 | |
| | GI:7438758 | | | | | |
| 7 | CAA41851.1 | 71 | 13 | | −19 | |
| | GI:32778 | | | | | |
| 8 | CAD60378.1 | 69 | 12 | | −16 | |
| | GI:27753335 | | | | | |
| 9 | BAC01458.1 | 71 | 12 | | −17 | |
| | GI:21668862 | | | | | |
| 10 | CAD60354.1 | 67 | 13 | | −18 | |
| | GI:27753287 | | | | | |
| 11 | AAC18279.1 | 72 | 13 | | −15 | ref - human |
| | GI:3170953 | | | | | antibody |
| 12 | CAD60400.1 | 69 | 11 | | −18 | |
| | GI:27753465 | | | | | |
| 13 | CAD60389.1 | 70 | 11 | | −18 | |
| | GI:27753357 | | | | | |
| 14 | CAD60364.1 | 69 | 13 | | −17 | |
| | GI:27753307 | | | | | |
| 15 | CAD60352.1 | 69 | 11 | | −18 | |
| | GI:27753283 | | | | | |
| 16 | CAD60349.1 | 71 | 13 | | −18 | |
| | GI:27753277 | | | | | |
| 17 | CAD66347.1 | 69 | 12 | | −18 | |
| | GI:27753273 | | | | | |
| 18 | CAD60300.1 | 66 | 14 | | −18 | |
| | GI:27753179 | | | | | |
| 19 | AAS85871.1 | 69 | 14 | | −16 | |
| | GI:46253798 | | | | | |
| 20 | CAF31294.1 | 72 | 8 | | −14 | Unpublished - |
| | GI:49523898 | | | | | probably |
| | | | | | | human |
| 21 | CAC28931.1 | 72 | 12 | | −18 | |
| | GI:12734096 | | | | | |
| 22 | CAA78572.1 | 71 | 12 | | −18 | |
| | GI:30966 | | | | | |
| 23 | CAA78561.1 | 70 | 12 | | −18 | |
| | GI:31019 | | | | | |
| 24 | ABA26208.1 | 70 | 10 | | −16 | |
| | GI:75707489 | | | | | |
| 25 | AAL96550.1 | 64 | 13 | | −22 | |
| | GI:19744554 | | | | | |
| 26 | AAZ94745.1 | 74 | 8 | 1-3 | −15 | no reference - |
| | GI:74039339 | | | | | human |
| | | | | | | therapeutic |
| | | | | | | library clone |
| 27 | AAB62913.1 | 71 | 9 | | −19 | |
| | GI:2253346 | | | | | |
| 28 | AAZ08900.1 | 70 | 14 | | −20 | |
| | GI:70798376 | | | | | |
| 29 | AAZ08886.1 | 70 | 12 | | −26 | |
| | GI:70798348 | | | | | |
| 30 | AAD53846.1 | 71 | 12 | | −19 | |
| | GI:5834160 | | | | | |
| 31 | CAC28912.1 | 72 | 13 | | −19 | |
| | GI:12734026 | | | | | |
| 32 | AAX19318.1 | 70 | 12 | | −20 | |
| | GI:60392056 | | | | | |
| 33 | CAC88717.1 | 73 | 13 | | −22 | |
| | GI:15886920 | | | | | |
| 34 | ABC67109.1 | 71 | 10 | | −17 | |
| | GI:84798320 | | | | | |
| 35 | ABC67046.1 | 71 | 11 | | −18 | |
| | GI:84798194 | | | | | |
| 36 | CAA75158.1 | 71 | 11 | | −22 | |
| | GI:4379079 | | | | | |
| 37 | BAC01328.1 | 62 | 11 | | −20 | |
| | GI:21668602 | | | | | |
| 38 | AAY57129.1 | 68 | 11 | | −23 | |
| | GI:66821321 | | | | | |
| 39 | AAY57128.1 | 68 | 11 | | −21 | |
| | GI:66821293 | | | | | |
| 40 | AAY57125.1 | 63 | 11 | | −23 | |
| | GI:66821255 | | | | | |
| 41 | AAV39619.1 | 68 | 11 | | −16 | |
| | GI:54778900 | | | | | |

The frameworks with the highest homology to the 4K21 heavy chain variable regions were hIgVH GMCSF01, 02, 05, and 26. All these antibodies have the same canonical structure as 4K21 heavy chain (1-3). The next large group of antibodies were very similar in their identity and similarity scores but two antibodies were selected base on their framework similarity scores. Framework similarity was calculated by analysing each residue in the framework region and the score is the number of non-identities. These two antibodies were hIgVH GMCSF11 and 20. The source of all the antibodies was checked and all were from human sources except for hIgVH GMCSF02 which was ignored. Hence, the antibodies selected with the highest homology to the variable heavy chain region of 4K21 were hIgVH GMCSF01, 05, 11, 20 and 26.

Grafting CDRS into Framework Sequences and Creation of Humanized Light and Heavy Chain Sequences

### Humanized 4K21 Light Chain

Four versions of a humanized 4K21 light chain variable region were created. FIG. 3 shows an alignment of these humanized versions of the 4K21 light chain antibody. Three of these, h4a, h4b and h4c were utilised in humanized antibodies described herein.

The humanized anti GM-CSF light chain, h4a (SEQ ID NO:9), is based on the hIgVL GMCSF02 and 05 frameworks—these two antibody light chains have an identical framework. Both antibodies were isolated from a library of therapeutic human antibodies. The library was developed from a mixture of human tonsil, umbilical cord, peripheral blood and bone marrow.

The humanized anti GM-CSF light chain, h4b (SEQ ID NO:10), is based on the hIgVL GMCSF06 framework. This antibody was isolated from the peripheral blood of a patient with chronic lymphocytic leukemia (Stamatopoulos et al. Blood 106(10):3575-3583, 2005)

The humanized anti GM-CSF light chain, h4c (SEQ ID NO:11), is based on the hIgVL GMCSF09 framework. This antibody was isolated from a library of therapeutic human antibodies. The library was developed from a mixture of human tonsil, umbilical cord, peripheral blood and bone marrow.

### Humanized 4K21 Heavy Chain

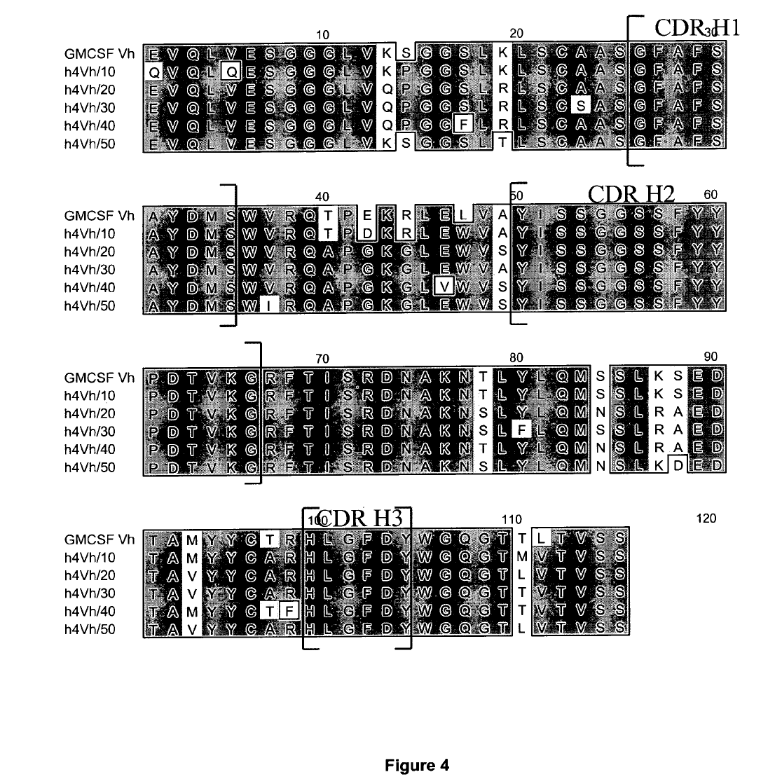

Five versions of a humanized 4K21 heavy chain variable region were created. FIG. 4 shows an alignment of these five humanized versions of the 4K21 heavy chain antibody.

The humanized anti GM-CSF heavy chain, h4/10 (SEQ ID NO:13), is based on the hIgVH GMCSF01. The antibody was isolated from a patient that was resistant to malarial infection (Lundquist et al., Infect. Immun. 74 (6): 3222-3231, 2006).

The humanized anti GM-CSF heavy chain, h4/20 (SEQ ID NO:14), is based on the hIgVH GMCSF05. This antibody was isolated from a library of therapeutic human antibodies. The library was developed from a mixture of human tonsil, umbilical cord, peripheral blood and bone marrow.

The humanized anti GM-CSF heavy chain, h4/30 (SEQ ID NO:15), is based on the hIgVH GMCSF11. The antibody was isolated from two healthy adults (Wang and Stollar, Clin. Immunol. 93 (2): 132-142, 1999).

The humanized anti GM-CSF heavy chain, h4/40 (SEQ ID NO:16), is based on the hIgVH GMCSF20. This antibody was isolated from a CD5+ B cell population.

The humanized anti GM-CSF heavy chain, h4/50 (SEQ ID NO:17), is based on the hIgVH GMCSF26. This human antibody was isolated from a patient with hairy cell leukemia. Note that the entry in the database did not have the complete sequence of framework 1. (The first 16 amino acids are missing.) To complete the framework one region the equivalent residues of the mouse heavy chain were inserted. All of these residues from the murine antibody are present in the human database except for the serine at amino acid 14. The serine at amino acid 14 seems to be unique to murine. Hence the inclusion of the serine at amino acid 14 would be regarded as a framework mutation back to the murine residue for this humanized version of the 4K21 heavy chain.

### Example 4

### Expression of Humanized Antibodies

Cloning Antibody Variable Region Genes into Vectors with Constant Region Gene

The heavy and light chain variable amino acid sequences were designed as described above. To produce antibodies containing these domains a DNA sequence encoding each variable region was optimised and synthesized by Geneart GmbH, Germany. The light chain variable gene had unique BsmB1 restriction sites at each end. The heavy chain gene had a BsmB1 site at the 5′ end and an Nhe1 site at the 3′ end. In addition, EcoRI and HindIII sites were added at the 5′ or 3′ end for ease of subcloning into other vectors.

To construct a full-length antibody gene the variable region gene was subcloned into a vector encoding a secretion signal and the constant domain. For the light chain, this vector contained the secretion signal sequence and the human constant kappa (CK) region gene separated by two unique BsmB1 sites. The heavy chain vectors contained the secretion signal and a human constant gamma (Cγ1) region gene separated by BsmB1 and Nhe1 sites.

The cloning process involved preparation of plasmid DNA by standard methods, digestion of the plasmid DNA with BsmB1 (light chain vector and Vk region gene) or BsmB1 and Nhe1 (heavy chain vector and Vh region gene) as recommended by the manufacturer (NEB), separation of DNA fragments by agarose gel electrophoresis, recovery of DNA fragments from the gel using a gel extraction kit (JetQuick, Genomed), ligation of variable gene fragment to vector fragment (T4 DNA ligase, NEB), transformation of DNA into competent E. coli cells (TOP10, Invitrogen). Plasmid DNA from transformed cells was analysed by restriction digest and the antibody gene in the plasmid was sequenced to confirm that the variable region had been subcloned in the correct reading frame.

Subcloning Antibody Genes into Expression Vector

After confirmation that the full-length antibody gene had the correct sequence it was subcloned into an expression vector. Examples of expression vectors that could be used include any of the pcDNA-, pLENTI-, pT-REX-, pAd-, pREP- or pCEP-mammalian expression vectors (Invitrogen), pTriEx1 or pBac vectors (Novagen), ZAP and pCMV expression vectors (Stratagene), GS expression system vectors (Lonza), pCMV5 cumate expression system vectors (Qbiogene), UCOE expression system plasmids (ML Laboratories) or MARtech expression plasmids (Selexis). In this instance the heavy chain genes (with HindIII site at 5′ end and EcoRI site at 3′ end) were subcloned into the HindIII-EcoRI sites downstream of the CMV promoter in a pEE6.4 vector (Lonza Biologics, GB). The light chain genes (with HindIII at 5′ end and EcoRI site at 3′ end) were subcloned into the HindIII-EcoR1 sites of pEE12.4 (Lonza Biologics, GB). The heavy chain expression cassette (with promoter, heavy chain coding sequence and polyadenylation signal and with NotI at 5′ end and BamHI at 3′ end) was subcloned into NotI-BamHI site downstream of the light chain expression cassette to create a single vector that expressed both heavy and light chains.

### Expressing Humanized Antibodies in Mammalian Cells

To express a humanized antibody, a heavy and light chain vector were, in some cases, cotransfected into CHO cells using lipofectamine (Invitrogen). Alternatively, the vector DNA could be transfected by electroporation, calcium phosphate precipitation, direct injection, gene gun or another method known to those skilled in the art. On most occasions a single vector encoding both heavy and light chains was transfected into CHO cells by electroporation.

### Transient Antibody Transfection and Expression.

On the day of transfection, 150 μg of DNA was transfected into 1.5×10⁷ CHO cells (at least 90% viable, Lonza Biologics, GB) with lipofectamine. Antibody expression was monitored by ELISA after 3-4 days.

### Purification of Humanized Antibodies

The transfected cells secrete antibody into the growth medium. Antibody was purified by protein G affinity chromatography. Fractions containing antibody, identified by SDS-PAGE or by human IgG-specific ELISA, were pooled. A human IgG-specific ELISA was used to determine the amount of antibody recovered and its concentration. Antibody purity was estimated by polyacrylamide gel electrophoresis.

### List of Humanized Antibodies Produced and Assayed

Table 6 below lists the different antibodies produced, showing the heavy and light chain sequences present in the antibodies.

TABLE 6

| Humanized monoclonal | Heavy chain | Light chain |
|---|---|---|
| antibody | sequences | sequences |
| hGM4/1 (h4K21-1) | h4/10, hG1 | h4a, hKa |
| hGM4/2 (h4K21-2) | h4/20, hG1 | h4a, hKa |
| hGM4/3 (h4K21-3) | h4/30, hG1 | h4a, hKa |
| hGM4/4 (h4K21-4) | h4/40, hG1 | h4a, hKa |
| hGM4/5 (h4K21-5) | h4/50, hG1 | h4a, hKa |
| hGM4/6 (h4K21-6) | h4/10, hG1 | h4b, hKa |
| hGM4/7 (h4K21-7) | h4/20, hG1 | h4b, hKa |
| hGM4/8 (h4K21-8) | h4/30, hG1 | h4b, hKa |
| hGM4/9 (h4K21-9) | h4/40, hG1 | h4b, hKa |
| hGM4/10 (h4K21-10) | h4/50, hG1 | h4b, hKa |
| hGM4/11 (h4K21-11) | h4/10, hG1 | h4c, hKa |
| hGM4/12 (h4K21-12) | h4/20, hG1 | h4c, hKa |
| hGM4/13 (h4K21-13) | h4/30, hG1 | h4c, hKa |
| hGM4/14 (h4K21-14) | h4/40, hG1 | h4c, hKa |
| hGM4/15 (h4K21-15) | h4/50, hG1 | h4c, hKa |

### Example 5

### Biological Properties of Humanized Antibodies

### Binding Affinity Analysis of Humanized Anti GM-CSF Antibodies

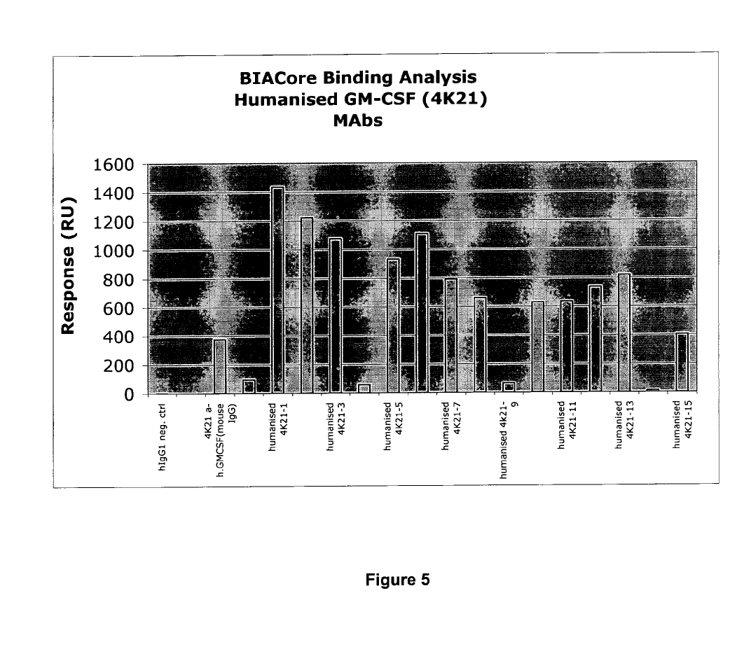

The binding affinity of the humanized antibodies was determined by BIACore analysis. Recombinant GM-CSF (Peprotech) at 100 μg/ml was prepared in sodium acetate, pH5, and immobilised to a CM5 sensor chip by amine coupling. Approximately 1200RU bound to the chip. The first experiment was to determine the binding activity of 15 humanized antibodies. These antibodies were prepared in HBS-EP at 1 μg/ml, injected for 3 minutes and compared to murine 4K21 and a commercially available anti GM-CSF antibody (BD). FIG. 5 shows that 12 of the 15 antibodies displayed equivalent, and in some cases up to 3.5 fold better binding to GM-CSF than murine 4K21.

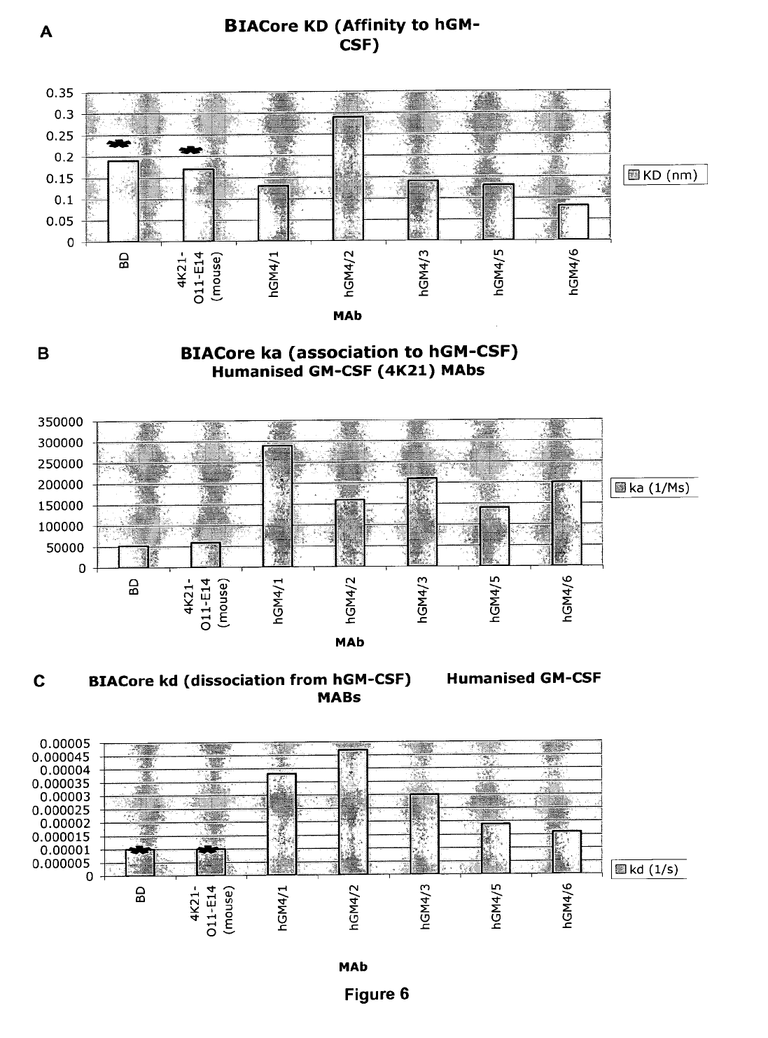

A second experiment was performed to determine the kinetics with which these antibodies bind to human GM-CSF. A range of antibody concentration was prepared in HBS-EP from 66.7 nM to 4.17 nM with 2-fold serial dilution. Again direct binding was quantitated using recombinant GM-CSF coupled to a CM5 chip—an uncoated flow cell was used as a reference. The antibody at each concentration was injected for 2 minutes with a stabilisation time of 10 minutes and dissociation time of 15 minutes. The BIACore kinetic analysis wizard was run to determine each antibody's association (ka [1/Ms]), dissociation (kd[1/s]) and affinity (KD [nM]) to GM-CSF. Table 7 and FIG. 6 shows that hGM4/1 has the highest association rate and that hGM4/6 has the highest affinity.

TABLE 7
Association rate, dissociation rate and binding
affinity of the GM-CSF humanized antibodies

| Antibody | ka (1/Ms) | kd (1/s) | KA (1/M) | KD (M) | KD (nm) | |
|---|---|---|---|---|---|---|
| BD | 5.20E+04 | <1e−5 | >5.2e9 | <1.9e−10 | <0.19 | |
| 4K21-O11-E14 | 5.90E+04 | <1e−5 | >5.9e9 | <1.7e−10 | <0.17 | |
| (mouse) | | | | | | |
| hGM4/1 | 2.90E+05 | 3.80E−05 | 7.70E+09 | 1.30E−10 | 0.13 | highest |
| | | | | | | association |
| hGM4/2 | 1.60E+05 | 4.70E−05 | 3.50E+09 | 2.90E−10 | 0.29 | |
| hGM4/3 | 2.10E+05 | 3.00E−05 | 7.10E+09 | 1.40E−10 | 0.14 | |
| hGM4/5 | 1.40E+05 | 1.90E−05 | 7.60E+09 | 1.30E−10 | 0.13 | |
| hGM4/6 | 2.00E+05 | 1.60E−05 | 1.20E+10 | 8.10E−11 | 0.081 | highest |
| | | | | | | affinity |

### Humanized Antibodies Block GM-CSF Mediated Cell Growth.

The GM-CSF neutralising activity of each antibody was determined with a TF-1 cell growth bioassay. TF-1 cells are dependant on the presence of GM-CSF for their growth and are maintained with 2 ng/ml recombinant GM-CSF (Peprotech). To perform the neutralization assay, a range of antibody concentrations was prepared in TF-1 growth media with 0.25 ng/ml GM-CSF. The antibody and GM-CSF were mixed at each concentration and incubated for 1 hr at 37° C. in a 96 well plate. Washed TF-1 cells (1×10⁵) were then added to each well and incubated at 37° C. for 72 hrs. Cellular growth was quantitated by pulsing each well for 4 hrs with 0.5 uCi of ³H-thymidine and the EC50 calculated. The antibodies were tested with concentrations of 400 nM to 28 fM with 3-fold serial dilution. As shown in Table 8, antibodies hGM4/1, hGM4/6 and hGM4/11 display good neutralizing activity.

TABLE 8
EC50 values for 4K21 and humanised antibodies as compared
to the non-GM-CSF specific hIgG1 antibody

| Antibody | EC50 |
|---|---|
| 4K21 n = 6 | 0.339 |
| hGM4/1 n = 4 | 12.423 |
| hGM4/2 n = 3 | 50.78 |
| hGM4/3 n = 2 | 12.705 |
| hGM4/5 n = 3 | 28.108 |
| hGM4/6 n = 4 | 7.95 |
| hGM4/7 n = 3 | 38.51 |
| hGM4/8 n = 2 | 26.377 |
| hGM4/9 n = 2 | 1472.975 |
| hGM4/10 n = 3 | 13.496 |
| hGM4/11 n = 4 | 5.862 |
| hGM4/12 n = 3 | 10.892 |
| hGM4/13 n = 3 | 16.409 |
| hGM4/15 n = 3 | 45.848 |
| hIgG1 n = 5 | |

### Example 6

### Back Mutations in the Heavy Chain Sequences of Humanized Antibodies and Properties of the Antibodies Generated

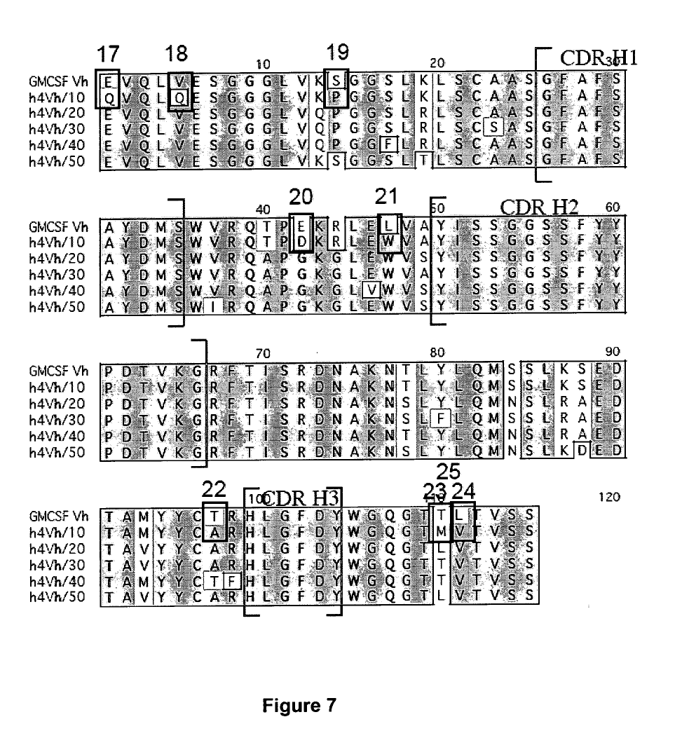

The antibodies (hGM4/1, hGM4/6 and hGM4/11) displaying the best neutralizing activity as illustrated in the TF-1 bioassay described in Example 5 above, all share the same heavy chain sequence (h4/10; SEQ ID NO:13). Without wishing to be bound by theory, it was speculated that at least a significant portion of the binding activity of the antibodies derives from this heavy chain. As can be seen from FIG. 7, the amino acid sequence of the h4/10 heavy chain framework differs at 8 positions compared to the murine framework (boxed in FIG. 7). A series of point back mutation and two double back mutations (back mutations to the corresponding murine sequence) were introduced into the h4/10 sequence at these sites to increase the binding affinity of the humanized anti GM-CSF antibody. The heavy chain sequences with the ‘back’ mutations were generated using site directed mutagenesis (Stratagene; in accordance with the manufacturer's instructions). These altered heavy chains were cloned into the antibody expression vector, transfected into CHO cells, expressed, purified by protein G affinity chromatography and quantitated as described above. Table 9 shows a list of the antibodies with altered frameworks and the ‘back’ mutation generated.

TABLE 9
Humanized antibodies with human framework ‘back’
mutations to murine residues.

| Humanized monoclonal | Heavy chain | Light chain |
|---|---|---|
| antibody | sequences | sequences |
| hGM4/17 | h4/10 Q1E, hG1 | h4c, hKa |
| hGM4/18 | h4/10 Q5V, hG1 | h4c, hKa |
| hGM4/19 | h4/10 P14S, hG1 | h4c, hKa |
| hGM4/20 | h4/10 D42E, hG1 | h4c, hKa |
| hGM4/21 | h4/10 W47L, hG1 | h4c, hKa |
| hGM4/22 | h4/10 A97T, hG1 | h4c, hKa |
| hGM4/23 | h4/10 M110T, hG1 | h4c, hKa |
| hGM4/24 | h4/10 V111L, hG1 | h4c, hKa |
| hGM4/25 | h4/10 M110T, V111L, hG1 | h4c, hKa |
| hGM4/34 | h4/10 W47L, A97T, hG1 | h4c, hKa |

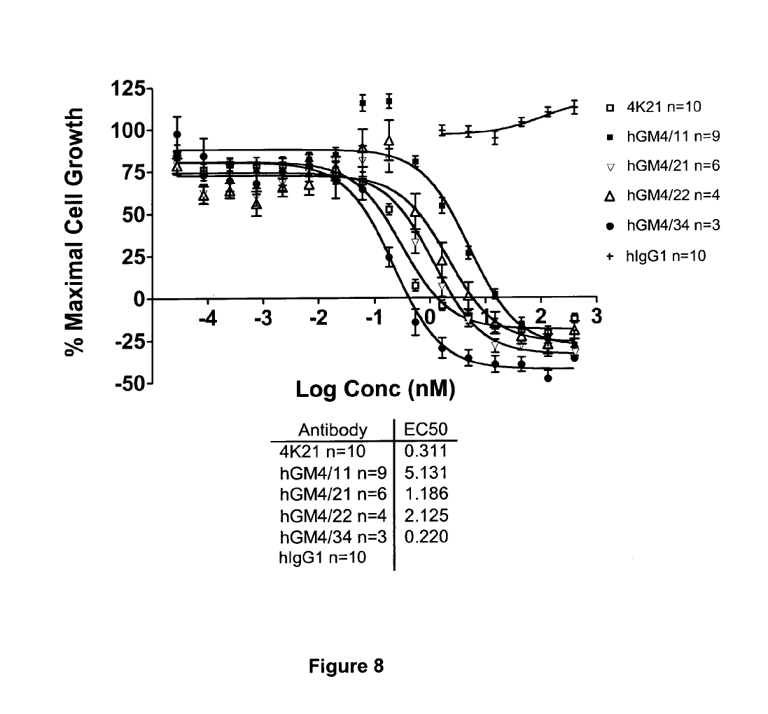

The neutralising activity of each of the antibodies listed in Table 9 was determined by TF-1 bioassay using the procedure as described above in Example 5. Three antibodies showed improved binding affinity than hGM4/11; hGM4/19, hGM4/21 and hGM4/22. Since the back mutations in hGM4/21 and hGM4/22 are located adjacent to CDR 2 and CDR 3 respectively, a double back mutation (W47L,A97T) was generated (hGM4/34). This antibody also displayed improved neutralizing ability, than the original 4K21 antibody (see FIG. 8).

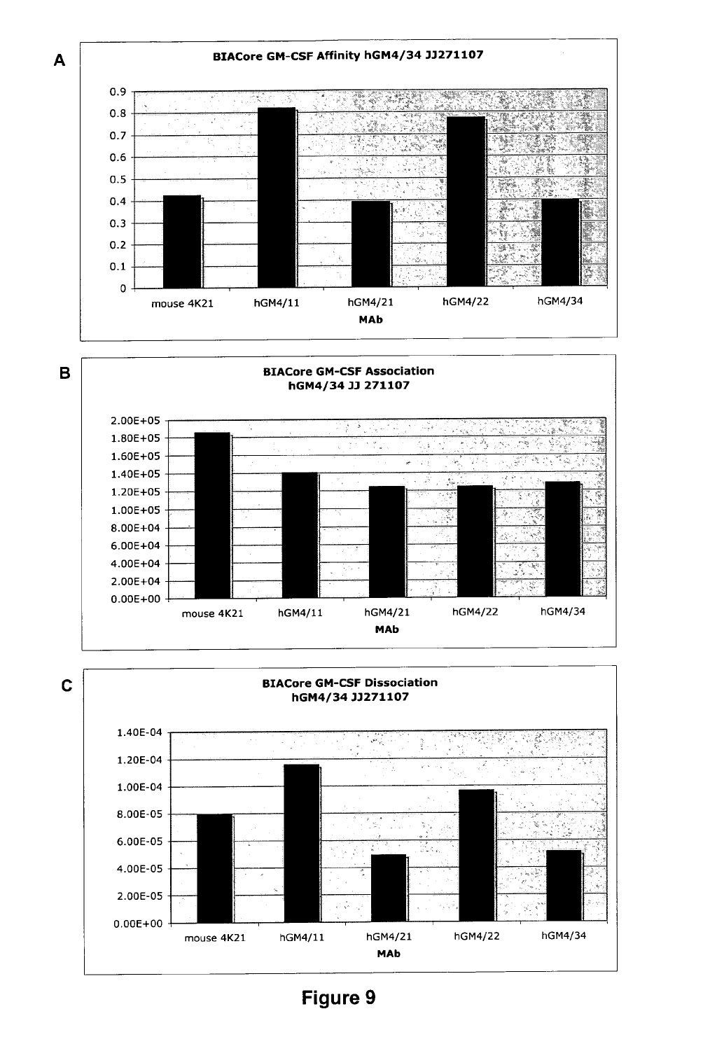

The binding kinetics of the antibodies listed in Table 9 was determined by BIAcore analysis. These kinetic assays were conducted using a protocol modified from that described above in Example 5. For the present kinetic analysis, the CM5 chip was prepared by immobilising either an anti mouse IgG or and anti human IgG. 4K21 or the humanized variants were then immobilised to the chip via the anti mouse IgG or the anti human IgG respectively. A range of GM-CSF concentrations were prepared in HBS-EP from 1000 nM to 15.63 nM with 2-fold serial dilution. The GM-CSF at each concentration was injected for 2 minutes and then dissociation measured over the next 10 minutes (flow rate 30 ul/min). The BIAcore kinetic analysis wizard was run to determine each antibody's association (ka[1/Ms]), dissociation (kd[1/s]) and affinity (KD[nM]). FIG. 9 shows that the affinity of hGM4/21 and hGM4/34 were the same as for 4K21.

1. An antibody or antigen-binding fragment thereof that binds to human GM-CSF or a fragment thereof, the antibody comprising a variable light chain region comprising the sequence as set forth in SEQ ID NO:1 or a fragment or variant thereof, and/or a variable heavy chain region comprising the sequence as set forth in SEQ ID NO:2 or a fragment or variant thereof.

2. (canceled)

3. The antibody or antigen-binding fragment of claim 1, wherein the antibody comprising a variable light chain region comprising the sequence as set forth in SEQ ID NO:1 or a fragment or variant thereof and a variable heavy chain sequence as set forth in SEQ ID NO:2 or a fragment or variant thereof.

4. The antibody or antigen-binding fragment of claim 1 wherein the antibody is a murine monoclonal antibody or a humanized derivative thereof which inhibits the activity of GM-CSF.

5. The antibody or antigen-binding fragment of claim 1 wherein the antibody is murine monoclonal antibody 4K21 deposited on 17 May 2007 at the European Collection of Cell Cultures (ECACC), Centre for Applied Microbiology and Research, Porton Down, Salisbury, United Kingdom, under Accession No. 07051601.

6. The antibody or antigen-binding fragment of claim 4, wherein the humanized derivative is a humanized form of murine monoclonal antibody 4K21 deposited at the European Collection of Cell Cultures (ECACC), Centre for Applied Microbiology and Research, Porton Down, Salisbury, United Kingdom, under Accession No. 07051601.

7. The humanized form of murine monoclonal antibody 4K21 as claimed in claim 6 wherein the variable light chain region comprises a sequence as set forth in any one of SEQ ID NOs:9 to 11 or a fragment or variant thereof, and/or the variable heavy chain region comprises a sequence as set forth in any one of SEQ ID NOs:13 to 15, 17 or 18 to 27, or a fragment or variant thereof.

8. (canceled)

9. The antibody or antigen-binding fragment of claim 1, wherein the antibody comprising within the light chain variable region at least one complementarity determining region (CDR) comprising a sequence as set forth in any one of SEQ ID Nos:3 to 5, and/or within the heavy chain variable region at least one complementarity determining region (CDR) comprising a sequence as set forth in any one of SEQ ID Nos:6 to 8, wherein the antibody or antigen-binding fragment thereof inhibits the activity of GM-CSF.

10-12. (canceled)

13. The antibody or antigen-binding fragment of claim 9, wherein the antibody comprising within the light chain variable region at least one complementarity determining region (CDR) comprising a sequence as set forth in any one of SEQ ID Nos:3 to 5 and within the heavy chain variable region at least one complementarity determining region (CDR) comprising a sequence as set forth in any one of SEQ ID Nos:6 to 8.

14. The antibody or antigen-binding fragment thereof as claimed in claim 9 wherein the antibody is a humanized antibody.

15. The humanized antibody or antigen-binding fragment thereof as claimed in claim 14 comprising a variable light chain region comprising the sequence as set forth in SEQ ID NO:9, SEQ ID NO:10 or SEQ ID NO:11 or a fragment or variant thereof, and/or a variable heavy chain region comprising the sequence as set forth in SEQ ID NO:13, SEQ ID NO:14, SEQ ID NO:15 or SEQ ID NO:17 or a fragment or variant thereof.

16. (canceled)

17. The humanized antibody or antigen-binding fragment thereof as claimed in claim 15 wherein the variable heavy chain region comprises the sequence as set forth in SEQ ID NO:13 or a fragment or variant thereof.

18. The humanized antibody or antigen-binding fragment thereof as claimed in claim 15 wherein the variant variable heavy chain region comprises one or more amino acid substitutions replacing amino acid residue(s) of the sequence set forth in SEQ ID NO:13, SEQ ID NO:14, SEQ ID NO:15 or SEQ ID NO:17 with amino acid residue(s) at the corresponding location(s) in the corresponding murine variable heavy chain region.

19. The humanized antibody or antigen-binding fragment thereof as claimed in claim 18 wherein the one or more amino acid substitutions are made to the variable heavy chain region comprising the sequence set forth in SEQ ID NO:13.

20. The humanized antibody or antigen-binding fragment thereof as claimed in claim 19 wherein the variant variable heavy chain region comprises a sequence as set forth in any one of SEQ ID NOs:18 to 27.

21. The humanized antibody or antigen-binding fragment thereof as claimed in claim 20 wherein the variant variable heavy chain region comprises the sequence as set forth in SEQ ID NO:27.

22. The humanized antibody or antigen-binding fragment thereof as of claim 14 wherein the humanized antibody comprises the variable light chain and heavy chain sequences selected from: SEQ ID NO: 9 and SEQ ID NO:13; SEQ ID NO:9 and SEQ ID NO:14; SEQ ID NO:9 and SEQ ID NO:15; SEQ ID NO:9 and SEQ ID NO:17; SEQ ID NO:10 and SEQ ID NO:13; SEQ ID NO:10 and SEQ ID NO:14; SEQ ID NO:10 and SEQ ID NO:15; SEQ ID NO:10 and SEQ ID NO:17; SEQ ID NO:11 and SEQ ID NO:13; SEQ ID NO:11 and SEQ ID NO:14; SEQ ID NO:11 and SEQ ID NO:15; SEQ ID NO:11 and SEQ ID NO:17; and SEQ ID NO:11 or any one of SEQ ID NOs:18 to 27.

23. The antibody or antigen-binding fragment thereof as claimed in claim 1 comprising a variable light chain region comprising an amino acid sequence having at least about 70% sequence identity to the amino acid sequence of SEQ ID NO: 1 and/or a variable heavy chain region comprising an amino acid sequence having at least about 70% identity to the amino acid sequence of SEQ ID NO:2.

24. A method for the treatment or prevention of a GM-CSF—mediated disease or condition or a disease or condition otherwise associated with elevated or aberrant GM-CSF expression and/or activity, the method comprising administering to a subject in need thereof an effective amount of at least one antibody or antigen-binding fragment thereof as claimed in any one of claim 1.

25. A pharmaceutical composition comprising one or more antibodies or antigen-binding fragments thereof as claimed in claim 1, optionally together with suitable pharmaceutically acceptable carriers and/or diluents.

26. The humanized antibody or antigen-binding fragment thereof as claimed in claim 15 comprising a variable light chain region comprising an amino acid sequence having at least about 70% identity to an amino acid sequence as set forth in SEQ ID NO:9, SEQ ID NO:10 or SEQ ID NO:11, and/or a variable heavy chain region comprising an amino acid sequence having at least about 70% identity to an amino acid sequence set forth in SEQ ID NO:13, SEQ ID NO:14, SEQ ID NO:15 or SEQ ID NO:17.


SEQUENCE LISTING (27 sequences)

SEQ ID NO: 1 - protein, 112 aa, Mus musculus
DVVMTQTPLSLPVSLGDQASISCRSSQSLVNSNGNTYLHWFLQKPGQSPKLLIYKVSNRFSGVPDRFSGSGSGTDFTLKISRVEAEDLGVYFCSQSTHVPPTFGGGTKLEIK

SEQ ID NO: 2 - protein, 115 aa, Mus musculus
EVQLVESGGGLVKSGGSLKLSCAASGFAFSAYDMSWVRQTPEKRLELVAYISSGGSSFYYPDTVKGRFTISRDNAKNTLYLQMSSLKSEDTAMYYCTRHLGFDYWGQGTTLTVSS

SEQ ID NO: 3 - protein, 16 aa, Mus musculus
RSSQSLVNSNGNTYLH

SEQ ID NO: 4 - protein, 7 aa, Mus musculus
KVSNRFS

SEQ ID NO: 5 - protein, 9 aa, Mus musculus
SQSTHVPPT

SEQ ID NO: 6 - protein, 10 aa, Mus musculus
GFAFSAYDMS

SEQ ID NO: 7 - protein, 17 aa, Mus musculus
YISSGGSSFYYPDTVKG

SEQ ID NO: 8 - protein, 6 aa, Mus musculus
HLGFDY

SEQ ID NO: 9 - protein, 112 aa, Mus musculus
DVVMTQSPLSLPVTLGQPASISCRSSQSLVNSNGNTYLHWFQQRPGQSPRRLIYKVSNRFSGVPDRFSGSGSGTDFTLKISRVEAEDVGVYYCSQSTHVPPTFGGGTKVEIK

SEQ ID NO: 10 - protein, 112 aa, Artificial
  Humanized sequence
DVVMTQSPLSLPVTLGQPASISCRSSQSLVNSNGNTYLHWFQQRPGQSPRRLIYKVSNRFSGVPDRFSGSGSGTDFTLKISRVEAEDVGVYYCSQSTHVPPTFGQGTKLEIK

SEQ ID NO: 11 - protein, 112 aa, Artificial
  Humanized sequence
DVVMTQSPLSLPVTPGEPASISCRSSQSLVNSNGNTYLHWYLQKPGQSPQLLIYKVSNRFSGVPDRFSGSGSGTDFTLKISRVEAEDVGVYYCSQSTHVPPTFGGGTKVEIK

SEQ ID NO: 12 - protein, 112 aa, Artificial
  Humanized sequence
DVVMTQSPLSLPVTPGEPASISCRSSQSLVNSNGNTYLHWYLQKPGQSPQLLIYKVSNRFSGVPDRFSGSGSGTDFTLKISRVEAEDVGVYYCSQSTHVPPTFGQGTKLEIK

SEQ ID NO: 13 - protein, 115 aa, Artificial
  Humanized sequence
QVQLQESGGGLVKPGGSLKLSCAASGFAFSAYDMSWVRQTPDKRLEWVAYISSGGSSFYYPDTVKGRFTISRDNAKNTLYLQMSSLKSEDTAMYYCARHLGFDYWGQGTMVTVSS

SEQ ID NO: 14 - protein, 115 aa, Artificial
  Humanized sequence
EVQLVESGGGLVQPGGSLRLSCAASGFAFSAYDMSWVRQAPGKGLEWVSYISSGGSSFYYPDTVKGRFTISRDNAKNSLYLQMNSLRAEDTAVYYCARHLGFDYWGQGTLVTVSS

SEQ ID NO: 15 - protein, 115 aa, Artificial
  Humanized sequence
EVQLVESGGGLVQPGGSLRLSCSASGFAFSAYDMSWVRQAPGKGLEWVAYISSGGSSFYYPDTVKGRFTISRDNAKNSLFLQMSSLRAEDTAVYYCARHLGFDYWGQGTTVTVSS

SEQ ID NO: 16 - protein, 115 aa, Artificial
  Humanized sequence
EVQLVESGGGLVQPGGFLRLSCAASGFAFSAYDMSWVRQAPGKGLVWVSYISSGGSSFYYPDTVKGRFTISRDNAKNTLYLQMNSLRAEDTAMYYCTFHLGFDYWGQGTTVTVSS

SEQ ID NO: 17 - protein, 115 aa, Artificial
  Humanized sequence
EVQLVESGGGLVKSGGSLTLSCAASGFAFSAYDMSWIRQAPGKGLEWVSYISSGGSSFYYPDTVKGRFTISRDNAKNSLYLQMNSLKDEDTAVYYCARHLGFDYWGQGTLVTVSS

SEQ ID NO: 18 - protein, 115 aa, Artificial
  Humanized sequence comprising murine back mutation
EVQLQESGGGLVKPGGSLKLSCAASGFAFSAYDMSWVRQTPDKRLEWVAYISSGGSSFYYPDTVKGRFTISRDNAKNTLYLQMSSLKSEDTAMYYCARHLGFDYWGQGTMVTVSS

SEQ ID NO: 19 - protein, 115 aa, Artificial
  Humanized sequence comprising murine back mutation
QVQLVESGGGLVKPGGSLKLSCAASGFAFSAYDMSWVRQTPDKRLEWVAYISSGGSSFYYPDTVKGRFTISRDNAKNTLYLQMSSLKSEDTAMYYCARHLGFDYWGQGTMVTVSS

SEQ ID NO: 20 - protein, 115 aa, Artificial
  Humanized sequence comprising murine back mutation
QVQLQESGGGLVKSGGSLKLSCAASGFAFSAYDMSWVRQTPDKRLEWVAYISSGGSSFYYPDTVKGRFTISRDNAKNTLYLQMSSLKSEDTAMYYCARHLGFDYWGQGTMVTVSS

SEQ ID NO: 21 - protein, 115 aa, Artificial
  Humanized sequence comprising murine back mutation
QVQLQESGGGLVKPGGSLKLSCAASGFAFSAYDMSWVRQTPEKRLEWVAYISSGGSSFYYPDTVKGRFTISRDNAKNTLYLQMSSLKSEDTAMYYCARHLGFDYWGQGTMVTVSS

SEQ ID NO: 22 - protein, 115 aa, Artificial
  Humanized sequence comprising murine back mutation
QVQLQESGGGLVKPGGSLKLSCAASGFAFSAYDMSWVRQTPDKRLELVAYISSGGSSFYYPDTVKGRFTISRDNAKNTLYLQMSSLKSEDTAMYYCARHLGFDYWGQGTMVTVSS

SEQ ID NO: 23 - protein, 115 aa, Artificial
  Humanized sequence comprising murine back mutation
QVQLQESGGGLVKPGGSLKLSCAASGFAFSAYDMSWVRQTPDKRLEWVAYISSGGSSFYYPDTVKGRFTISRDNAKNTLYLQMSSLKSEDTAMYYCTRHLGFDYWGQGTMVTVSS

SEQ ID NO: 24 - protein, 115 aa, Artificial
  Humanized sequence comprising murine back mutation
QVQLQESGGGLVKPGGSLKLSCAASGFAFSAYDMSWVRQTPDKRLEWVAYISSGGSSFYYPDTVKGRFTISRDNAKNTLYLQMSSLKSEDTAMYYCARHLGFDYWGQGTTVTVSS

SEQ ID NO: 25 - protein, 115 aa, Artificial
  Humanized sequence comprising murine back mutation
QVQLQESGGGLVKPGGSLKLSCAASGFAFSAYDMSWVRQTPDKRLEWVAYISSGGSSFYYPDTVKGRFTISRDNAKNTLYLQMSSLKSEDTAMYYCARHLGFDYWGQGTMLTVSS

SEQ ID NO: 26 - protein, 115 aa, Artificial
  Humanized sequence comprising murine back mutation
QVQLQESGGGLVKPGGSLKLSCAASGFAFSAYDMSWVRQTPDKRLEWVAYISSGGSSFYYPDTVKGRFTISRDNAKNTLYLQMSSLKSEDTAMYYCARHLGFDYWGQGTTLTVSS

SEQ ID NO: 27 - protein, 115 aa, Artificial
  Humanized sequence comprising murine back mutation
QVQLQESGGGLVKPGGSLKLSCAASGFAFSAYDMSWVRQTPDKRLELVAYISSGGSSFYYPDTVKGRFTISRDNAKNTLYLQMSSLKSEDTAMYYCTRHLGFDYWGQGTMVTVSS